(19) United States
(12) Patent Application Publication
(10) Pub. No.: US 2016/0310029 A1
(43) Pub. Date: Oct. 27, 2016

(54) METHOD AND APPARATUS FOR DETERMINING A PREMATURE VENTRICULAR CONTRACTION IN A MEDICAL MONITORING DEVICE

(71) Applicant: Medtronic, Inc., Minneapolis, MN (US)

(72) Inventor: Shantanu Sarkar, Roseville, MN (US)

(21) Appl. No.: 14/694,847

(22) Filed: Apr. 23, 2015

(51) Int. Cl. A61B 5/04; A61B 5/00; A61B 5/0456

(52) U.S. Cl. CPC A61B 5/04014; A61B 5/0456; A61B 5/686

(57) ABSTRACT

A method and medical monitoring device for determining the occurrence of a premature ventricular contraction that includes sensing a cardiac signal and determining R-waves in response to the sensed cardiac signal, determining RR intervals between the determined R-waves, determining whether a first interval criteria is satisfied in response to the determined intervals, determining a correlation between the determined R-waves, determining whether a first correlation criteria is satisfied in response to the determined correlation, and determining the premature ventricular contraction is occurring in response to the first interval criteria and the first correlation criteria being satisfied.

### TECHNICAL FIELD

The disclosure relates generally to a medical monitoring device for monitoring a cardiac signal, and more particular, to a medical monitoring device for monitoring the occurrence of premature ventricular contractions in a cardiac signal to evaluate cardiac condition.

### BACKGROUND

Numerous implantable medical devices are available for acute or chronic implantation within patients. Some implantable medical devices may be used to chronically monitor physiological signals of the patient, such as implantable hemodynamic monitors, implantable cardiac monitors (sometimes referred to as implantable loop recorders or ECG monitors), implantable blood chemistry monitors, implantable pressure monitors, or the like. Other implantable devices may be configured to deliver a therapy in conjunction with or separate from the monitoring of physiological signals.

Premature ventricular contractions (PVCs) may occur frequently in healthy individuals and may be caused by caffeine, smoking, alcohol consumption, stress, exhaustion, pharmacological toxicity, among other causes. An implantable monitoring device capable of detecting PVC burden may be a useful tool in some instances to evaluate cardiac wellness, perform risk stratification for sudden cardiac death or evaluate safety in pharmaceutical trials. Therefore, what is needed is an implantable monitoring device for monitoring occurrence of premature ventricular contractions to evaluate cardiac condition.

### BRIEF DESCRIPTION OF THE DRAWINGS

FIG. 1 is a conceptual diagram illustrating an exemplary implantable monitoring device system that may be used to monitor one or more physiological parameters of a patient according to the present disclosure.

FIG. 2 is a functional block diagram of an exemplary implantable monitoring device system that may be used to monitor one or more physiological parameters of a patient according to the present disclosure.

FIG. 3 is a schematic diagram illustrating detecting a premature ventricular contraction according to an embodiment of the present disclosure.

FIG. 4 is a flowchart of a method for detecting a premature ventricular contraction in a medical monitoring device according to an embodiment of the disclosure.

FIG. 5 is a flowchart of a method for detecting a premature ventricular contraction in a medical monitoring device according to an embodiment of the disclosure.

### DETAILED DESCRIPTION

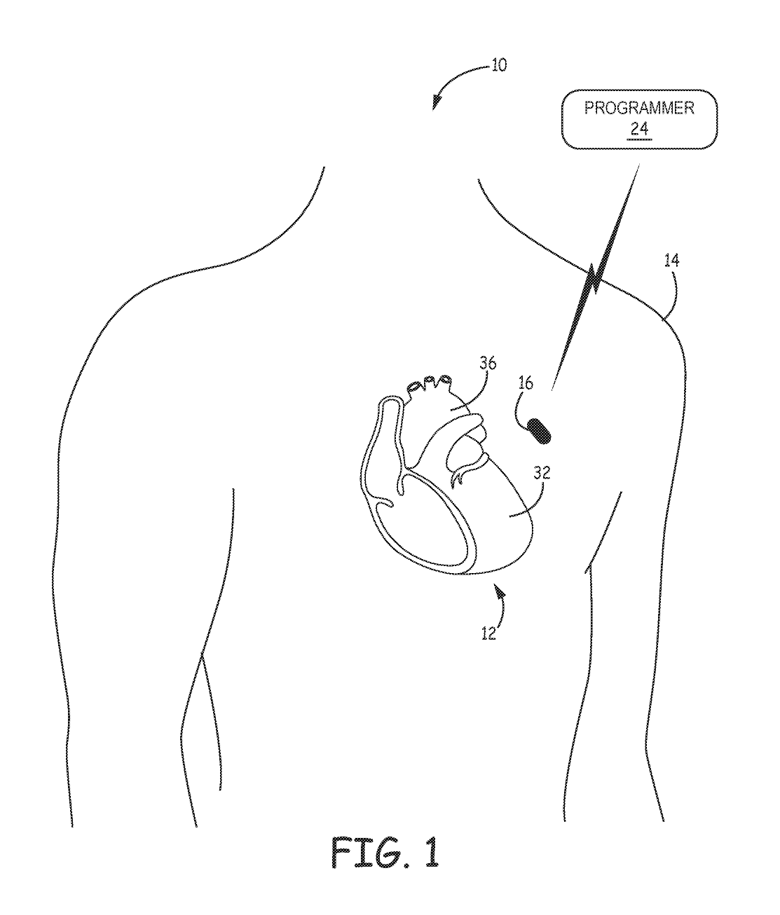

FIG. 1 is a conceptual diagram illustrating an exemplary implantable monitoring device system that may be used to monitor one or more physiological parameters of a patient according to the present disclosure. As illustrated in FIG. 1, an implantable monitoring device system 10 includes an implantable medical device (IMD) 16, which is coupled to programmer 24. IMD 16 may be a subcutaneous sensing device configured to sense signals indicative of one or more physiological parameters of patient 14. For example, IMD 16 may sense and/or store electrocardiogram (ECG) signals. In some examples, IMD 16 may be configured to sense ECG or other signals and detect arrhythmias, e.g., ventricular and/or supra-ventricular arrhythmias, based on the signals. An example of a monitoring device in the which the present disclosure may be utilized is described, for example, in U.S. Pat. No. 5,987,352 to Klein et al., incorporated herein by reference in it's entirety.

Although the examples described herein include IMD 16 configured to sense physiological signals of patient 14, in other examples IMD 16 may alternatively or additionally be configured to deliver therapy to patient 14. For example, IMD 16 may be an implantable leadless pacemaker that provides electrical signals to heart 12 via one or more electrodes (not shown in FIG. 1) on its outer housing, such as described, for example, in U.S. patent application Ser. No. 14/520,847, incorporated herein by reference in it's entirety. Additionally or alternatively, IMD 16 may sense electrical signals attendant to the depolarization and repolarization of heart 12 via electrodes on its outer housing. In some examples, IMD 16 provides therapy to patient 14 based on sensed physiological signals.

In some examples, IMD 16 may be configured to be implanted proximate to heart 12, e.g., as illustrated in FIG. 1. In other examples, IMD 16 may be configured to be implanted proximate to or within another portion of the body of patient 14.

In the examples described herein, IMD 16 includes a header, which may include one or more components of IMD 16, and may be formed separately from the rest of IMD 16. In some examples, the header may include an antenna, at least one of the one or more electrodes, and/or an attachment plate configured to attach the header to another portion of IMD 16. The header may be formed via the molding techniques described herein.

In the example of FIG. 1, IMD 16 is positioned subcutaneously in a left pectoral region of patient 14. In other examples, however, IMD 16 may be positioned within any suitable region of patient 14. In some examples, depending on the location of implant, IMD 16 may include other sensing and/or stimulation functionalities. For example, IMD 16 may provide atrioventricular nodal stimulation, fat pad stimulation, vagal stimulation, or other types of neurostimulation, and/or may sense one or more parameters of heart 12 or another parameter of patient 12. In some examples, system 10 may include a plurality of leadless IMDs 16, e.g., to provide stimulation and/or sensing at a variety of locations.

FIG. 1 further depicts programmer 24 in communication with IMD 16. In some examples, programmer 24 comprises a handheld computing device, computer workstation, or networked computing device. Programmer 24 includes a user interface that presents information to and receives input from a user. It should be noted that the user may also interact with programmer 24 remotely via a networked computing device.

A user, such as a physician, technician, surgeon, electrophysiologist, other clinician, or patient, interacts with programmer 24 to communicate with IMD 16. For example, the user may interact with programmer 24 to retrieve physiological or diagnostic information from IMD 16. A user may also interact with programmer 24 to program IMD 16, e.g., select values for operational parameters of the IMD 16. For example, the user may use programmer 24 to retrieve information from IMD 16 regarding the rhythm of heart 12, trends therein over time, or arrhythmic episodes.

IMD 16 and programmer 24 may communicate via wireless communication using any techniques known in the art. Examples of communication techniques may include, for example, low frequency or radiofrequency (RF) telemetry, proximal inductive interaction, or tissue conductance communication, but other techniques are also contemplated. In some examples, programmer 24 may include a programming head that may be placed proximate to or in contact with the patient's body near the IMD 16 implant site in order to improve the quality or security of communication between IMD 16 and programmer 24.

Although the examples described herein refer to leadless IMD 16, IMD 16 may alternatively be coupled to one or more leads comprising one or more electrodes configured to sense the one or more physiological parameters of patient 14 and/or to deliver the therapy to heart 12 of patient 14. Additionally, although the examples herein describe monitoring physiological signals via IMD 16, IMD 16 may additionally or alternatively be configured for pacing therapy for heart 12, neurostimulation therapy, defibrillation therapy, or cardioversion therapy via one or more electrodes of system 10.

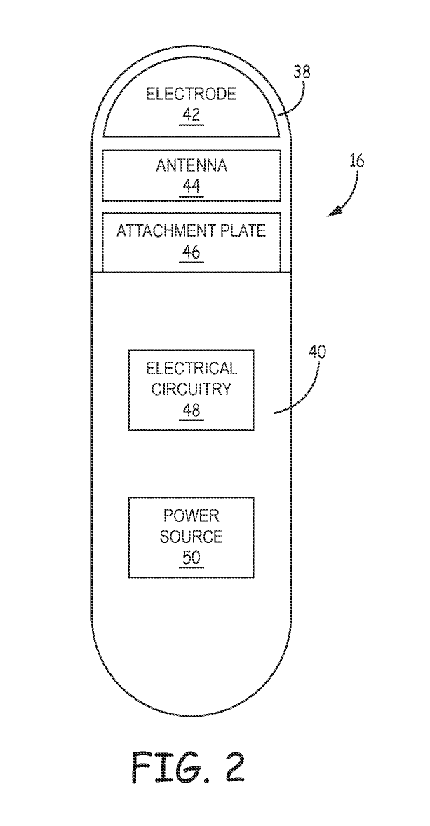

FIG. 2 is a functional block diagram of an exemplary implantable monitoring device system that may be used to monitor one or more physiological parameters of a patient according to the present disclosure. As illustrated in FIG. 2, IMD 16 may include header 38 coupled to body portion 40. In the examples described herein, header 38 may include electrode 42, antenna 44, and attachment plate 46. In particular, electrode 42, antenna 44, and attachment plate 46 may be molded into header 38 via a two-shot molding process. Body portion 40 of IMD 16 may include electrical circuitry 48 and power source 50, in some examples, which may be contained within a hermetic housing or can, e.g., formed of titanium or ceramic.

As shown in FIG. 2, header 38 includes at least one electrode 42. Electrode 42 may be configured to sense physiological signals of patient 14 and/or to deliver electrical stimulation therapy to patient 14, e.g., to treat a cardiac disorder of patient 14. IMD 16 may sense signals or deliver stimulation via electrode 42 in combination with another electrode, such as the housing of body portion 40. In some examples, electrode 42 may be coated with a material configured to improve performance, e.g., sensing or pacing performance. For example, electrode 42 may be coated with a conductive material such as Titanium Nitride (TiN).

Header 38 also includes antenna 44. Antenna 44 may be configured to transmit and/or receive electromagnetic signals for communication. For example, antenna 44 may be configured to transmit to and/or receive signals from programmer 24. Antenna 44 may be coupled to electrical circuitry 48 of IMD 16, which may drive antenna 44 to transmit signals to programmer 24, and may receive signals received from programmer 24 via antenna 44. In the example shown in FIG. 2, header 38 additionally includes attachment plate 46, which is configured to mechanically couple header 38 to body portion 40 of IMD 16, as will be described in further detail below.

In the example shown in FIG. 2, body portion 40 of IMD 16 is configured to house electrical circuitry 48 and power source 50. Electrical circuitry 48 may comprise one or more electrical circuits configured to perform any function of IMD 16. For example, the electrical circuitry 48 may be coupled to antenna 44 to receive and/or transmit signals. Electrical circuitry 48 may additionally or alternatively be configured to analyze physiological signals, e.g., signals sensed via electrode 42, and/or to control delivery of stimulation or other therapies. Body portion 40 is also configured to house power source 50, which may be configured to provide energy to various components of IMD 16, such as electrical circuitry 48.

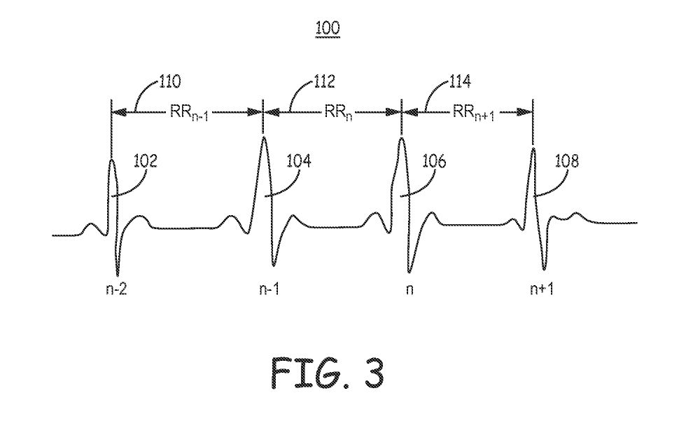

FIG. 3 is a schematic diagram illustrating detecting a premature ventricular contraction according to an embodiment of the present disclosure. As illustrated in FIG. 3, a cardiac signal 100 may be sensed by a monitoring device, such as a subcutaneous monitoring device, for example, from which the device identifies R-waves 102-108. In order to determine whether one of the R-waves 104-106 is associated with the occurrence of a premature ventricular contraction, the device determines interval differences between intervals formed by the detected R-waves 104-108 and a morphology criterion between the R-waves 104-108. For example, the device determines an RR interval 110 associated with the first detected R-wave 104 and a previously detected R-wave 102, an interval 112 associated with the second detected R-wave 106 and the previously detected R-wave 104, and an interval 114 associated with the third R-wave 108 and the previously detected R-wave 106. A premature ventricular contraction is then identified by comparing the differences between the RR intervals 110-114, as described below.

In addition to determining the interval differences to identify a premature ventricular contraction, the device also determines a morphology criterion that is used in combination with the interval differences to ascertain whether one of the intervals 104-108 is the result of a premature ventricular contraction being sensed. For example, the device determines a correlation between the morphology of the first R-wave 104 and the second R-wave 106, between the first R-wave 104 and the third R-wave 108, and between the second R-wave 106 and the third R-wave, using known correlation determination techniques. For example, a difference-sum technique may be utilized in which instead of multiplying, the baseline, which can be computed as the median of the waveform from first R-wave to last R-wave under consideration, is subtracted from the two waveforms, and a point by point difference is determined, and a sum of the difference is used to determine the correlation or match between the two waveforms. Two highly correlated waveforms will lead to a lower difference sum compared to two waveforms that are not highly correlated. According to another embodiment, wavelet based template matching is used to determine a correlation between the R-waves 104-108, such as described for example in U.S. Pat. No. 6,393,316 to Gillberg et al., incorporated herein by reference in it's entirety. During wavelet based template matching, two segments of waveforms, the template and the current beat are matched (same as cross-correlated) with each other using wavelet coefficients at three different lags, with the best match representing he match score (or cross-correlation).

According to another embodiment, a filter technique made be utilized to determine the correlation between the R-waves 104-108 in which one of the waveforms is used as filter coefficients of a digital FIR filter, and then the other waveform filtered using the first waveform as filter coefficients with the maximum output after the filtering being the best match (or cross-correlation) value. In yet another exemplary embodiment, a feature set matching technique may be utilized to determine correlation of the R-waves 104-108 in which rather than multiplying each point of the waveform, features are decoded from each waveform, such as amplitude maximum, amplitude minimum, amplitude mean and median, number of samples within a percentage of median, polarity (based on difference of max or min from mean value), r-wave width, r-wave area under curve. The feature set is then compared between the two waveforms to generate a match score, or correlation. It is understood that any known method for determining a correlation between R-waves may be utilized in order to carry out the determination of the occurrence of a premature ventricular contraction according to the present disclosure.

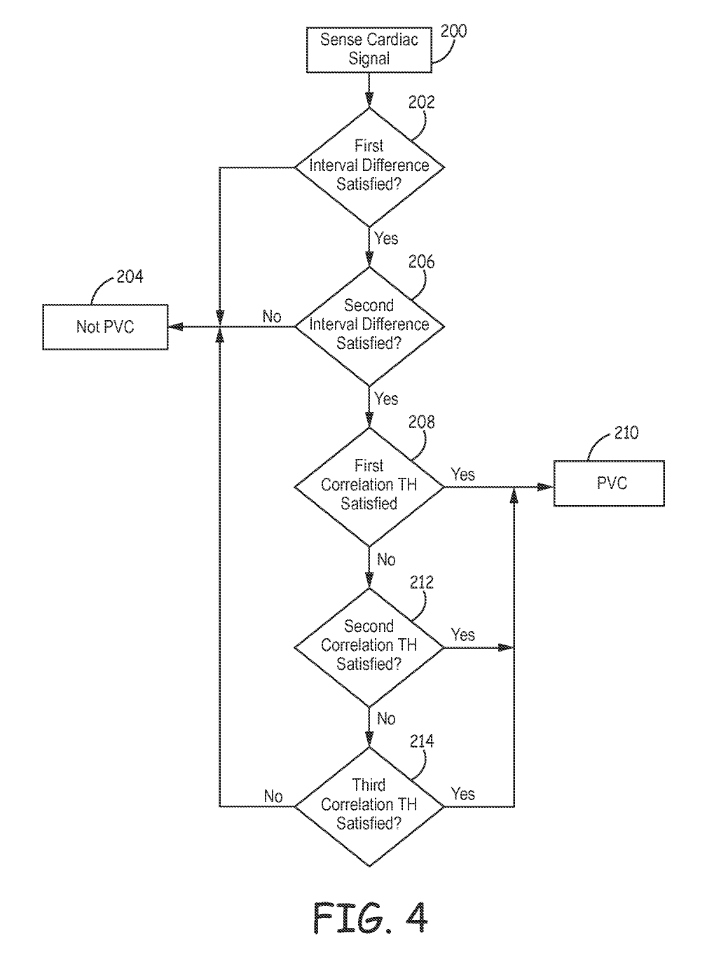

FIG. 4 is a flowchart of a method for detecting a premature ventricular contraction in a medical monitoring device according to an embodiment of the disclosure. As illustrated in FIGS. 3 and 4, the device senses the cardiac signal 100, Block 200, and determines R-waves 102-108 associated with the signal 100. The device determines whether a first interval difference associated with the R-waves is satisfied, Block 202, as described below. If the first interval difference is not satisfied, No in Block 202, a premature ventricular contraction is determined not to occur, Block 204. If the first interval difference threshold is satisfied, Yes in Block 202, the device determines whether a second interval difference is satisfied, Block 206. If the second interval difference is not satisfied, No in Block 206, a premature ventricular contraction is determined not to occur, Block 204. If the second interval difference is satisfied, Yes in Block 206, the device determines whether a first correlation threshold between the R-waves 104-108 is satisfied, Block 208.

If the first correlation threshold is satisfied, Yes in Block 208, a premature ventricular contraction is determined to have occurred, Block 210. If the first correlation threshold is not satisfied, No in Block 208, the device determines whether a second correlation threshold is satisfied, Block 212, described below. If the second correlation threshold is satisfied, Yes in Block 212, a premature ventricular contraction is determined to have occurred, Block 210. If the second correlation threshold is not satisfied, No in Block 212, the device determines whether a third correlation threshold is satisfied, Block 214, described below. If the third correlation threshold is satisfied, Yes in Block 214, a premature ventricular contraction is determined to have occurred, Block 210. If the third correlation threshold is not satisfied, No in Block 214, the device determines a premature ventricular contraction is determined not to occur, Block 204.

In this way, the device combines RR interval criteria with morphologic criteria to determine the presence of a ventricular contraction by determining the premature ventricular contraction when both the first and second interval difference thresholds are satisfied, Yes in Blocks 202 and 206, and at least one of the first, second or third correlation thresholds have been satisfied, Yes in Block 208, 212 or 214.

According to an embodiment of the disclosure, in order to determine whether the first interval difference is satisfied, the device compares the RR-interval 112 associated with the second R-wave 106 (RRₙ) with both the RR-interval 104 associated with the first R-wave 104 (RRₙ₋₁) and the RR-interval 114 associated with the third R-wave 108 (RRₙ₊₁). A determination is made as to whether the RR-interval 112 associated with the second R-wave 106 (RRₙ) is shorter than the RR-interval 104 associated with the first R-wave 104 (RRₙ₋₁) with the difference being greater than an interval threshold, such as being greater than 10 ms, for example. If the difference is determined to be greater than the difference threshold, the first interval difference is determined to be satisfied, Yes in Block 202.

Similarly, a determination is made as to whether the RR-interval 112 associated with the second R-wave 106 (RRₙ) is shorter than the RR-interval 114 associated with the third R-wave 104 (RRₙ₊₁) with the difference being greater than an interval threshold, such as being greater than 10 ms, for example. If the difference is determined to be greater than the difference threshold, the second interval difference is determined to be satisfied, Yes in Block 206.

According to an embodiment of the disclosure, in order to determine whether the correlation thresholds 208-212 are satisfied, the device determines correlations between the R-waves 104-108 using known correlation schemes. In order to determine whether the first correlation threshold is satisfied, Block 208, a determination is made as to whether a first correlation (C_{n-1,n+1}) between the first R-wave 104 and the third R-wave 108 is greater than a correlation threshold, such as 0.9 for example. In addition, the device determines whether a difference between the first correlation and a second correlation (C_{n-1,n}) determined between the first R-wave 104 and the second R-wave 106 is greater than a correlation difference threshold, such as 0.1 for example, and whether a difference between the first correlation and a third correlation (C_{n,n+1}) between the second R-wave 106 and the third R-wave is greater than the correlation difference threshold. If the first correlation between the first R-wave 104 and the third R-wave 108 is greater than the correlation threshold, and both the difference between the first correlation and the second correlation and the difference between the first correlation and the third correlation is greater than the correlation difference threshold, the first correlation threshold is determined to be satisfied, Yes in Block 208.

According to an embodiment of the disclosure, in order to determine whether the second correlation threshold is satisfied, Block 212, a determination is made as to whether a first correlation (C_{n-1,n+1}) between the first R-wave 104 and the third R-wave 108 is greater than a correlation threshold, such as 0.8 for example. In addition, the device determines whether a difference between the first correlation and a second correlation (C_{n-1,n}) determined between the first R-wave 104 and the second R-wave 106 is greater than a correlation difference threshold, such as 0.3 for example, and whether a difference between the first correlation and a third correlation between the second R-wave 106 and the third R-wave is greater than the correlation difference threshold. If the first correlation between the first R-wave 104 and the third R-wave 108 is greater than the correlation threshold, and both the difference between the first correlation and the second correlation and the difference between the first correlation and the third correlation is greater than the correlation difference threshold, the second correlation threshold is determined to be satisfied, Yes in Block 212.

Similarly, in order to determine whether the third correlation threshold is satisfied, Block 214, a determination is made as to whether a first correlation (C_{n-1,n+1}) between the first R-wave 104 and the third R-wave 108 is greater than a correlation threshold, such as 0.7 for example. In addition, the device determines whether a difference between the first correlation and a second correlation (C_{n-1,n}) determined between the first R-wave 104 and the second R-wave 106 is greater than a correlation difference threshold, such as 0.6 for example, and whether a difference between the first correlation and a third correlation (C_{n,n+1}) between the second R-wave 106 and the third R-wave is greater than the correlation difference threshold. If the first correlation between the first R-wave 104 and the third R-wave 108 is greater than the correlation threshold, and both the difference between the first correlation and the second correlation and the difference between the first correlation and the third correlation is greater than the correlation difference threshold, the third correlation threshold is determined to be satisfied, Yes in Block 214.

In this way, the device determines the RR-interval criteria by evaluating the three RR-intervals 110-114 using the following evaluation criteria:

RRₙ<RRₙ₋₁−10 ms and RRₙ<RRₙ₊₁−10 ms Criteria 1:

where RRₙ is the RR-interval 112 associated with the second R-wave 106, RRₙ₋₁ is the RR-interval 110 associated with the first R-wave 104, and RRₙ₊₁ is the RR-interval 114 associated with the third R-wave 108. In addition, the device determines the correlation criteria by evaluating the correlation of the three R-waves 104-108 using the following correlation criteria:

{C_{n-1,n+1}>0.9 and C_{n-1,n+1}−C_{n-1,n}>0.1 and C_{n-1,n+1}−C_{n,n+1}>0.1} OR {C_{n-1,n+1}>0.8 and C_{n-1,n+1}−C_{n-1,n}>0.3 and C_{n-1,n+1}−C_{n,n+1}>0.3} OR {C_{n-1,n+1}>0.7 and C_{n-1,n+1}−C_{n-1,n}>0.6 and C_{n-1,n+1}−C_{n,n+1}>0.6} Criteria 2:

where C_{n-1,n+1} is the cross-correlation between the first R-wave 104 and the third R-wave 108, C_{n-1,n} is the cross correlation between first R-wave 104 and the second R-wave 106, C_{n,n+1} is the cross correlation between the second R-wave 106 and the third R-wave 108.

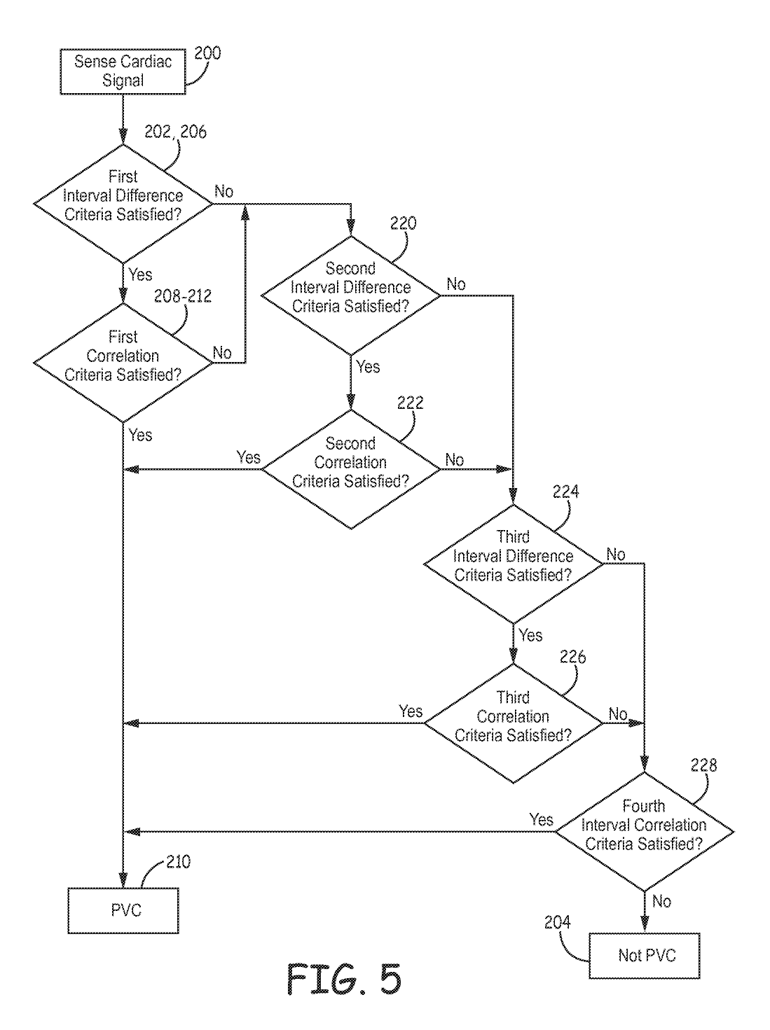

FIG. 5 is a flowchart of a method for detecting a premature ventricular contraction in a medical monitoring device according to an embodiment of the disclosure. According to an embodiment of the present disclosure, in order to improve sensitivity of the determination of the occurrence of a premature ventricular contraction, additional interval and correlation criteria may be utilized in addition to criteria 1 and criteria 2, described above. For example, as illustrated in FIG. 5, if either one of the first interval difference or the second interval difference is not satisfied (No in Block 202 or Block 206 of FIG. 4), additional interval criteria Block 220 and correlation criteria Block 222 may be utilized to determine the occurrence of a premature ventricular contraction. For example, according to one embodiment, the device may determine whether a ratio of the RR-interval associated with the second R-wave 106 and the RR-interval associated with first R-wave 104 (RRₙ/RRₙ₋₁) is less than an RR-interval ratio threshold, such as 1.25 for example, and whether a ratio of the RR-interval associated with the second R-wave 106 and the RR-interval associated with third R-wave 108 (RRₙ/RRₙ₊₁) is less than the RR-interval ratio threshold. In addition, a determination is made as to whether the RR-interval 212 associated with the second R-wave 106 (RRₙ) is less than a secondary interval threshold, such as 800 ms for example. If both the RR-interval ratio threshold and the secondary interval threshold are satisfied, the additional interval criteria 220 is determined to be satisfied, Yes in Block 220, the device determines whether the secondary correlation threshold is satisfied, Block 222.

In this way, the device further determines the RR-interval criteria by evaluating the three RR-intervals 110-114 using the following secondary interval evaluation criteria:

RRₙ/RRₙ₋₁<1.25 and RRₙ/RRₙ₊₁<1.25 and RRₙ<800 ms Criteria 3:

According to one embodiment, in order to determine whether the secondary correlation is satisfied, Block 222, the device determines whether one of two secondary correlation thresholds are satisfied. For example, a first secondary correlation is satisfied if a correlation (C_{n-1,n+1}) between the first R-wave 104 and the third R-wave 108 is greater than a first secondary correlation threshold, such 0.98, for example, and both a correlation (C_{n-1,n}) between the first R-wave 104 and the second R-wave 106 and a correlation (C_{n,n+1}) between the second R-wave 106 and the third R-wave 208 is less than a second secondary threshold, such as 0.75 for example. A second secondary correlation is satisfied if a correlation (C_{n-1,n+1}) between the first R-wave 104 and the third R-wave 108 is greater than a first secondary correlation threshold, such 0.95, for example, and both a correlation (C_{n-1,n}) between the first R-wave 104 and the second R-wave 106 and a correlation (C_{n,n+1}) between the second R-wave 106 and the third R-wave 208 is less than a second secondary threshold, such as 0.55 for example. In this way, the device further determines the RR-interval criteria by evaluating the three R-waves 104-108 using the following secondary correlation evaluation criteria:

{C_{n-1,n+1}>0.98 and C_{n-1,n}<0.75 and C_{n,n+1}<0.75} OR {C_{n-1,n+1}>0.95 and C_{n-1,n}<0.55 and C_{n,n+1}<0.55} Criteria 4:

If one of the first and the second secondary correlation criteria are satisfied, Yes in Block 222, and a premature ventricular contraction is determined to be identified, Block 210. If either the secondary interval criteria are not satisfied, No in Block 220, or if both the first and second secondary correlation criteria are not satisfied, No in Block 222, a premature ventricular contraction is not determined to occur, Block 204.

According to another embodiment, in order to further improve sensitivity the device may determine if third RR-interval and correlation criteria are satisfied. For example, if either the secondary interval criteria are not satisfied, No in Block 220, or if both the first and second secondary correlation criteria are not satisfied, No in Block 222, the device may determine additional interval criteria Block 224 and correlation criteria, Block 226, to determine the occurrence of a premature ventricular contraction. For example, according to one embodiment, the device may determine whether the RR-interval 112 associated with the second R-wave 106 (RRₙ) is less than a first RR-interval threshold, such as 400 ms for example, and both the R-interval 110 associated with the first R-wave 104 (RRₙ₋₁) and the RR-interval 114 associated with the third R-wave 108 (RRₙ₊₁) are greater than a second interval threshold, such as 500 ms for example.

In this way, the device further determines the RR-interval criteria by evaluating the three RR-intervals 110-114 using the following third interval evaluation criteria:

RRₙ<400 ms and RRₙ₋₁>500 ms and RRₙ₊₁>500 ms: Criteria 5:

If the RR-interval 112 associated with the second R-wave is less than the first RR-interval threshold, and both the R-interval 110 associated with the first R-wave 104 and the RR-interval 114 associated with the third R-wave 108 are greater than a second interval threshold, the third interval criteria is determined to be satisfied, Yes in Block 224, and the device determines whether the third correlation criteria are met, Block 226.

According to an embodiment of the present disclosure, in order to determine whether the third correlation criteria are met, Block 226, the device determines whether a first correlation (C_{n-1,n+1}) between the first R-wave 104 and the third R-wave is greater than a third correlation threshold, such as 0.5 for example. In addition, the device determines whether a difference between the first correlation and a second correlation (C_{n-1,n}) determined between the first R-wave 104 and the second R-wave 106 is greater than a correlation threshold, such as 0.1 for example, and whether a difference between the first correlation and a third correlation (C_{n,n+1}) between the second R-wave 106 and the third R-wave is greater than the correlation difference threshold. In this way, the device further determines the RR-interval criteria by evaluating the three R-waves 104-108 using the following third correlation evaluation criteria:

C_{n-1,n+1}>0.5 and C_{n-1,n+1}−C_{n-1,n}>0.1 and C_{n-1,n+1}−C_{n,n+1}>0.1 Criteria 6:

If the first correlation between the first R-wave 104 and the third R-wave 108 is greater than the correlation threshold, and both the difference between the first correlation and the second correlation and the difference between the first correlation and the third correlation is greater than the correlation difference threshold, the third correlation threshold is determined to be satisfied, Yes in Block 226, and therefore a premature ventricular contraction is determined to occur, Block 210. On the other hand, if the first correlation between the first R-wave 104 and the third R-wave 108 is not greater than the correlation threshold, or either the difference between the first correlation and the second correlation or the difference between the first correlation and the third correlation is not greater than the correlation difference threshold, the third correlation threshold is not determined to be satisfied, No in Block 226, and therefore a premature ventricular contraction is not determined to occur, Block 204.

According to another embodiment, in order to further improve sensitivity the device may determine if fourth RR-interval criteria are satisfied. For example, if either the third interval criteria are not satisfied, No in Block 224, or if either one of the first and second third correlation criteria are not satisfied, No in Block 226, the device may determine additional interval criteria Block 228 to determine the occurrence of a premature ventricular contraction. For example, according to one embodiment, the device may determine whether a ratio of the RR-interval associated with the second R-wave 106 and the RR-interval associated with first R-wave 104 (RRₙ/RRₙ₋₁) is less than an RR-interval ratio threshold, such as 0.65 for example, and whether a ratio of the RR-interval associated with the second R-wave 106 and the RR-interval associated with third R-wave 108 (RRₙ/RRₙ₊₁) is less than then RR-interval ratio threshold. In addition, a determination is made as to whether the RR-interval 212 associated with the second R-wave 106 (RRₙ) is less than a secondary interval threshold, such as 400 ms for example.

In this way, the device further determines the RR-interval criteria by evaluating the three RR-intervals 110-114 using the following fourth interval evaluation criteria:

RRₙ/RRₙ₋₁<0.65 and RRₙ/RRₙ₊₁<0.65 and RRₙ<400 ms Criteria 7:

If both the RR-interval ratio threshold and the secondary interval threshold are satisfied, the fourth additional interval criteria is determined to be satisfied, Yes in Block 228, and the device determines that a premature ventricular contraction has occurred, Block 210. On the other hand, if either the RR-interval ratio threshold or the secondary interval threshold are not satisfied, the fourth additional interval criteria is determined not to be satisfied, no in Block 228, and the device determines that a premature ventricular contraction has not occurred, Block 204.

The method in principle can be extended to detect couplets (two consecutive premature ventricular intervals) or triplets (three consecutive ventricular intervals). In the case of a couplet, one would consider four RR intervals RRₙ₋₂, RRₙ₋₁, RRₙ, and RRₙ₊₁, with RRₙ₋₁, RRₙ, representing the couplet. All the RR interval criteria, namely criteria 1, 3, 5, 7, with RRₙ₋₂, RRₙ₋₁, replacing RRₙ₋₁, RRₙ, in the criteria and in the all the criteria where RRₙ is compared to an interval threshold will be replaced by comparing both RRₙ₋₁, RRₙ, to that same interval threshold. For example the modified criteria 7 could be

RRₙ₋₁/RRₙ₋₂<0.65 and RRₙ₋₁/RRₙ₊₁<0.65 and RRₙ<400 ms and RRₙ₋₁<400 ms Couplet criteria 7:

Similarly, for correlation thresholds, C_{n-1,n} will be replaced by C_{n-2,n-1} and C_{n,n+1} will be replaced by C_{n-1,n+1}. Additionally, a new correlation criteria which compares the correlation between the couplet, C_{n-1,n}, will be compared to a correlation threshold and optionally added to criteria 2, 4, and 6. For example criteria 6 may be modified to

C_{n-2,n+1}>0.5 and C_{n-1,n}>0.5 and C_{n-2,n+1}−C_{n-2,n-1}>0.1 and C_{n-2,n-1-1}−C_{n-1,n+1}>0.1 Couplet criteria 6:

Once a premature ventricular contraction has been determined to occur, Block 210, the device may perform any one of or a number of tasks, such as storing the occurrence of premature ventricular contractions for future upload to an external device, storing the premature ventricular contractions and correlating the information with intermittent symptoms the patient may be experiencing to determine patient condition, using the information to provide a patient premature ventricular contraction burden diagnostic, such as the number that occur per day or per hour. The burden diagnostic may be utilized for risk stratification of the patient, such as for sudden cardiac death, for example, to identify patients who may be eligible for an EP study, which may then lead to a determination of the need for an implantable cardiac device, or to an ablation procedure being performed. In addition, the detection of the occurrence of premature ventricular contractions can be used as a computational device, such as for determining heart rate turbulence, or may be used to augment other detection algorithms, such as determining not to use such beats when determining respiration rate from ECG amplitude, or to reduce falsely detecting cardiac events, such atrial fibrillation, for example.

Thus, an apparatus and method have been presented in the foregoing description with reference to specific embodiments. It is appreciated that various modifications to the referenced embodiments may be made without departing from the scope of the invention as set forth in the following claims.

We claim:

1. A method of determining occurrence of a premature ventricular contraction in a medical device, comprising:
sensing a cardiac signal and determining R-waves in response to the sensed cardiac signal;
determining RR intervals between the determined R-waves;
determining whether a first interval criteria is satisfied in response to the determined intervals;
determining a correlation between the determined R-waves;
determining whether a first correlation criteria is satisfied in response to the determined correlation; and
determining the premature ventricular contraction is occurring in response to the first interval criteria and the first correlation criteria being satisfied.

2. The method of claim 1, wherein determining whether a first interval criteria is satisfied comprises:
determining a first RR interval associated with a first R-wave of the determined R-waves, a second RR interval associated with a second R-wave of the determined R-waves, and a third RR interval associated with a third R-wave of the determined R-waves;
comparing the second RR interval with the first RR interval to generate a first interval difference;
determining whether the first interval difference is greater than an interval difference threshold;
comparing the second RR interval with the third RR interval to generate a second interval difference; and
determining whether the second interval difference is greater than the interval difference threshold.

3. The method of claim 2, further comprising determining the premature ventricular contraction is not occurring in response to one of the first interval difference and the second interval difference not being greater than the interval difference threshold.

4. The method of claim 1, wherein determining whether a first correlation criteria is satisfied in response to the determined correlation comprises:
determining a first R-wave of the determined R-waves, a second R-wave of the determined R-waves, and a third R-wave of the determined R-waves;
determining a first correlation between the first R-wave and the third R-wave;
determining whether the first correlation is greater than a first correlation threshold;
determining a second correlation between the first R-wave and the second R-wave;
determining whether a first difference between the first correlation and the second correlation is greater than a first correlation difference threshold;
determining a third correlation between the second R-wave and the third R-wave;
determining whether a second difference between the first correlation and the third correlation is greater than the first correlation difference threshold; and
determining a premature ventricular contraction is occurring in response to the first correlation being greater than the first correlation threshold and both the first difference and the second difference being greater than the first correlation difference threshold.

5. The method of claim 4, wherein determining whether a first correlation criteria is satisfied in response to the determined correlation comprises:
determining whether the first correlation is greater than a second correlation threshold;
determining whether the first difference between the first correlation and the second correlation is greater than a second correlation difference threshold;
determining whether the second difference between the first correlation and the third correlation is greater than the second correlation difference threshold; and
determining a premature ventricular contraction is occurring in response to the first correlation being greater than the second correlation threshold and both the first difference and the second difference being greater than the second correlation difference threshold.

6. The method of claim 5, wherein determining whether a first correlation criteria is satisfied in response to the determined correlation comprises:
determining whether the first correlation is greater than a third correlation threshold;
determining whether the first difference between the first correlation and the second correlation is greater than a third correlation difference threshold;
determining whether the second difference between the first correlation and the third correlation is greater than the third correlation difference threshold; and
determining a premature ventricular contraction is occurring in response to the first correlation being greater than the third correlation threshold and both the first difference and the second difference being greater than the third correlation difference threshold.

7. The method of claim 6, wherein the first correlation threshold is 0.9, the second correlation threshold is 0.8, the third correlation threshold is 0.7, the first correlation difference threshold is 0.1, the second correlation difference threshold is 0.3 and the third correlation difference threshold is 0.6.

8. The method of claim 1, further comprising:
determining, in response to one of the first interval criteria and the first correlation criteria not being satisfied, whether a second interval criteria is satisfied in response to the determined intervals;
determining, in response to the second interval criteria being satisfied, whether a second correlation criteria is satisfied in response to the determined correlation; and
determining the premature ventricular contraction is occurring in response to the second interval criteria and the second correlation criteria being satisfied.

9. The method of claim 8, wherein determining whether a second interval criteria is satisfied in response to the determined intervals comprises:
determining a first RR interval associated with a first R-wave of the determined R-waves, a second RR interval associated with a second R-wave of the determined R-waves, and a third RR interval associated with a third R-wave of the determined R-waves;
determining whether a ratio of the second RR interval and the first RR interval is greater than an interval ratio threshold;
determining whether a ratio of the second RR interval and the third RR interval is greater than the interval ratio threshold; and
determining whether the second RR interval is less than a first interval threshold.

10. The method of claim 9, wherein determining whether a second correlation criteria is satisfied in response to the determined correlation comprises:
determining a first R-wave of the determined R-waves, a second R-wave of the determined R-waves, and a third R-wave of the determined R-waves;
determining a first correlation between the first R-wave and the third R-wave;
determining whether the first correlation is greater than a first correlation threshold;
determining a second correlation between the first R-wave and the second R-wave;
determining whether the second correlation is less than a second correlation threshold;
determining a third correlation between the second R-wave and the third R-wave; and
determining whether the third correlation is less than the second correlation threshold.

11. The method of claim 10, further comprising:
determining whether a third interval criteria is satisfied in response to the determined intervals;
determining, in response to the third interval criteria being satisfied, whether a third correlation criteria is satisfied in response to the determined correlation; and
determining the premature ventricular contraction is occurring in response to the third interval criteria and the third correlation criteria being satisfied.

12. The method of claim 11, wherein determining whether a third interval criteria is satisfied in response to the determined intervals comprises:
determining whether the second RR interval is less than a second interval threshold;
determining whether the first RR interval is greater than a third interval threshold; and
determining whether the third RR interval is greater than the third interval threshold.

13. The method of claim 12, wherein determining whether a third correlation criteria is satisfied in response to the determined correlation comprises:
determining whether the first correlation is greater than a third correlation threshold;
determining whether a difference between the first correlation and the second correlation is greater than a difference threshold; and
determining whether a difference between the first correlation and the third correlation is greater than the difference threshold.

14. The method of claim 1, further comprising:
determining a first RR interval associated with a first R-wave of the determined R-waves, a second RR interval associated with a second R-wave of the determined R-waves, and a third RR interval associated with a third R-wave of the determined R-waves;
determining, in response to one of the first interval criteria and the first correlation criteria not being satisfied, whether a ratio of the second RR interval and the first RR interval is less than an interval ratio threshold;
determining whether a ratio of the second RR interval and the third RR interval is less than the interval ratio threshold; and
determining whether the second RR interval is less than an interval threshold.

15. A medical monitoring device for determining the occurrence of a premature ventricular contraction, comprising:
a sensing electrode sensing a cardiac signal; and
a processor configured to determine R-waves in response to the sensed cardiac signal, determine RR intervals between the determined R-waves, determine whether a first interval criteria is satisfied in response to the determined intervals, determine a correlation between the determined R-waves, determine whether a first correlation criteria is satisfied in response to the determined correlation, and determine the premature ventricular contraction is occurring in response to the first interval criteria and the first correlation criteria being satisfied.

16. The medical monitoring device of claim 15, wherein the processor is configured to determine a first RR interval associated with a first R-wave of the determined R-waves, a second RR interval associated with a second R-wave of the determined R-waves, and a third RR interval associated with a third R-wave of the determined R-waves, compare the second RR interval with the first RR interval to generate a first interval difference, determine whether the first interval difference is greater than an interval difference threshold, compare the second RR interval with the third RR interval to generate a second interval difference, and determine whether the second interval difference is greater than the interval difference threshold.

17. The medical monitoring device of claim 16, wherein the processor is configured to determine the premature ventricular contraction is not occurring in response to one of the first interval difference and the second interval difference not being greater than the interval difference threshold.

18. The medical monitoring device of claim 15, wherein the processor is configured to determine a first R-wave of the determined R-waves, a second R-wave of the determined R-waves, and a third R-wave of the determined R-waves, determine a first correlation between the first R-wave and the third R-wave, determine whether the first correlation is greater than a first correlation threshold, determine a second correlation between the first R-wave and the second R-wave, determine whether a first difference between the first correlation and the second correlation is greater than a first correlation difference threshold, determine a third correlation between the second R-wave and the third R-wave, determine whether a second difference between the first correlation and the third correlation is greater than the first correlation difference threshold, and determine a premature ventricular contraction is occurring in response to the first correlation being greater than the first correlation threshold and both the first difference and the second difference being greater than the first correlation difference threshold.

19. The medical monitoring device of claim 18, wherein the processor is further configured to determine whether the first correlation is greater than a second correlation threshold, determine whether the first difference between the first correlation and the second correlation is greater than a second correlation difference threshold, determine whether the second difference between the first correlation and the third correlation is greater than the second correlation difference threshold, and determine a premature ventricular contraction is occurring in response to the first correlation being greater than the second correlation threshold and both the first difference and the second difference being greater than the second correlation difference threshold.

20. The medical monitoring device of claim 19, wherein the processor is configured to determine whether the first correlation is greater than a third correlation threshold, determine whether the first difference between the first correlation and the second correlation is greater than a third correlation difference threshold, determine whether the second difference between the first correlation and the third correlation is greater than the third correlation difference threshold, and determine a premature ventricular contraction is occurring in response to the first correlation being greater than the third correlation threshold and both the first difference and the second difference being greater than the third correlation difference threshold.

21. The medical monitoring device of claim 20, wherein the first correlation threshold is 0.9, the second correlation threshold is 0.8, the third correlation threshold is 0.7, the first correlation difference threshold is 0.1, the second correlation difference threshold is 0.3 and the third correlation difference threshold is 0.6.

22. The medical monitoring device of claim 15, wherein the processor is configured to determine, in response to one of the first interval criteria and the first correlation criteria not being satisfied, whether a second interval criteria is satisfied in response to the determined intervals, determine, in response to the second interval criteria being satisfied, whether a second correlation criteria is satisfied in response to the determined correlation, and determine the premature ventricular contraction is occurring in response to the second interval criteria and the second correlation criteria being satisfied.

23. The medical monitoring device of claim 22, wherein the processor is configured to determine a first RR interval associated with a first R-wave of the determined R-waves, a second RR interval associated with a second R-wave of the determined R-waves, and a third RR interval associated with a third R-wave of the determined R-waves, determine whether a ratio of the second RR interval and the first RR interval is greater than an interval ratio threshold, determine whether a ratio of the second RR interval and the third RR interval is greater than the interval ratio threshold, and determine whether the second RR interval is less than a first interval threshold.

24. The medical monitoring device of claim 23, wherein the processor is further configured to determine a first R-wave of the determined R-waves, a second R-wave of the determined R-waves, and a third R-wave of the determined R-waves, determine a first correlation between the first R-wave and the third R-wave, determine whether the first correlation is greater than a first correlation threshold, determine a second correlation between the first R-wave and the second R-wave, determine whether the second correlation is less than a second correlation threshold, determine a third correlation between the second R-wave and the third R-wave, and determine whether the third correlation is less than the second correlation threshold.

25. The medical monitoring device of claim 24, wherein the processor is configured to determine whether a third interval criteria is satisfied in response to the determined intervals, determine, in response to the third interval criteria being satisfied, whether a third correlation criteria is satisfied in response to the determined correlation, and determine the premature ventricular contraction is occurring in response to the third interval criteria and the third correlation criteria being satisfied.

26. The medical monitoring device of claim 25, wherein the processor is configured to determine whether the second RR interval is less than a second interval threshold, determine whether the first RR interval is greater than a third interval threshold, and determine whether the third RR interval is greater than the third interval threshold.

27. The medical monitoring device of claim 26, wherein the processor is configured to determine whether the first correlation is greater than a third correlation threshold, determine whether a difference between the first correlation and the second correlation is greater than a difference threshold, and determine whether a difference between the first correlation and the third correlation is greater than the difference threshold.

28. The medical monitoring device of claim 15, wherein the processor is configured to determine a first RR interval associated with a first R-wave of the determined R-waves, a second RR interval associated with a second R-wave of the determined R-waves, and a third RR interval associated with a third R-wave of the determined R-waves, determine, in response to one of the first interval criteria and the first correlation criteria not being satisfied, whether a ratio of the second RR interval and the first RR interval is less than an interval ratio threshold, determine whether a ratio of the second RR interval and the third RR interval is less than the interval ratio threshold, and determine whether the second RR interval is less than an interval threshold.

29. The medical monitoring device of claim 15, wherein the medical monitoring device comprises a subcutaneous monitoring device.

30. A non−transitory, computer-readable storage medium storing instructions for causing a processor included in a medical monitoring device to perform a method for determining occurrence of a premature ventricular contraction, the method comprising:
sensing a cardiac signal and determining R-waves in response to the sensed cardiac signal;
determining RR intervals between the determined R-waves;
determining whether a first interval criteria is satisfied in response to the determined intervals;
determining a correlation between the determined R-waves;
determining whether a first correlation criteria is satisfied in response to the determined correlation; and
determining the premature ventricular contraction is occurring in response to the first interval criteria and the first correlation criteria being satisfied.
